(19) United States
(12) Patent Application Publication
(10) Pub. No.: US 2025/0366803 A1
(43) Pub. Date: Dec. 4, 2025

(54) RADIATION SHIELD ASSEMBLY AND TRAY THEREFOR

(71) Applicant: Norad Designs LLC, Oviedo, FL (US)

(72) Inventors: John Barry EGOLF, Oviedo, FL (US); Scott POLLAK, Winter Park, FL (US)

(73) Assignee: Norad Designs LLC, Oviedo, FL (US)

(21) Appl. No.: 19/222,800

(22) Filed: May 29, 2025

Related U.S. Application Data
(60) Provisional application No. 63/653,341, filed on May 30, 2024.

(51) Int. Cl. A61B 6/10

(52) U.S. Cl. CPC A61B 6/107

(57) ABSTRACT

A radiation shield assembly, including a tray therefor, are disclosed. The tray has a base portion configured to be positioned underneath at least a portion of a patient, a first side coupled to the base portion at a first non-zero angle, a second side positioned opposite the first side and coupled to the base portion at a second non-zero angle, and a cranial side coupled to the base portion at a third non-zero angle. The radiation shield assembly further includes a first radiation attenuating material that shields radiation, a plurality of connectors for connecting the radiation attenuating material to the first side, the second side, and the cranial side, a second radiation attenuating material that shields radiation, and a plurality of second connectors for removably coupling the second radiation attenuating material to the tray.

### TECHNICAL FIELD

This disclosure relates generally to a radiation shield assembly and a tray for a radiation shield assembly.

### BACKGROUND

Radiation exposure is detrimental to human health. For example, a comprehensive review of available biologic and biophysical data supports a “no-threshold” risk model for radiation exposure since the risk of cancer may increase linearly at low doses of radiation without a threshold. The dose of radiation has the potential to cause a small increased risk of malignancy in humans. (National Research Council. Health Risks from Exposure to Low Levels of Ionizing Radiation: BEIR VII Phase 2. Washington, D.C.: National Academies, 2006.).

For example, within the survivors of the Hiroshima and Nagasaki atomic bombings, which represents a large population that includes all ages and both sexes, more than 60% of exposed survivors received a dose of radiation of less than 100 mSv (the definition of low dose used by the BEIR VII report). (National Research Council, 2006.) The Radiation Effects Research Foundation (RERF) in Japan has conducted follow-up studies on these survivors for more than 50 years to evaluate the health effects of ionizing radiation. From these studies, it was found that the occurrence of solid cancers increases in proportion to radiation dose. (Preston D L, Ron E, Tokuoka S, et al. Solid cancer incidence in atomic bomb survivors: 1958-1998. Radiat Res, 2007; 168: 1-64., Cullings H M, Fujita S, Funamoto S, et al. Dose estimation for atomic bomb survivor studies: Its evolution and present status. Radat Res, 2006; 166: 219-54.) See also, Sanchez R., Vano E, Fernandez J M, Gallejo J J. Staff radiation doses in real-time display inside the angiography room. Cardiovasc InterventRadiol, 2010.

Many different medical radiologic procedures or examinations, such as electrophysiological procedures, cardiac catheterization, angioplasty, cardiac stenting, cardiac valve procedures, and orthopedic procedures require the use of radiation (e.g., ionizing radiation, etc.). Although many different technologies attempt to avoid or minimize radiation during these procedures, there is still a moderate to high x-ray exposure as evidenced by reported fluoroscopy in numerous studies. (Cano O, Alonso P, Osca J, et al. Initial experience with a new image integration module designed for reducing radiation exposure during electrophysiological ablation procedures. J Cardiovasc Electrophysial, 2015; 26: 662-670., Valderrabano M, Greenberg S, Razavi H, et al. 3D cardiovascular navigation system: accuracy and reduction in radiation exposure in left ventricular lead implant. J Cardiovasc Electrophysiol, 2014; 25: 87-93.) Implant procedures may incur a higher exposure to the practitioner since the x-ray generator may be closer to the practitioner.

Some technology allows real-time assessment of radiation dose exposure at a given location. In radiation protection dosimetry, two types of dosimeters may be used: passive and active (direct reading). Passive dosimeters, such as film badges, may integrate the radiation dose over the measurement period. Active electronic dosimeters may combine a detector with the readout to display the radiation dose value (e.g., the rate of radiation exposure). (Ankerhold U, Hupe O, Ambrosi P. Deficiencies of active electronic radiation protection dosimeters in pulsed fields. Oxford University Press, 2009; 135:149-153.) Real-time radiation dose feedback utilizing dosimeters have been shown to reduce radiation exposure to the practitioners. (Racadio J, Nachabe R, Carelson B, et al. Effect of real-time radiation dose feedback on pediatric interventional radiology stop radiation exposure. Journal of Vascular and Interventional Radiology, 2013; 25:119-126.).

During a radiologic procedure, a radiation source, such as an x-ray tube below the table holding the patient, may emit radiation (e.g., x-rays) as a direct radiation beam toward an area of the patient's body that is intended to be examined. Most of the direct radiation beam enters into the patient in order to allow the patient to be examined and subsequently exits the patient's body. The area of the patient's body that is under examination receives some radiation due to the direct radiation beam. The entrance radiation dose is the amount of radiation that enters into the patient and the exit radiation dose is the amount of radiation that exits from the patient.

However, radiation from the direct radiation beam deflects, which causes the radiation to scatter and forms “scatter radiation.” Scatter radiation refers to any radiation that is outside of the direct radiation beam. A portion of the radiation may scatter before and/or after the radiation enters into and exits from the patient's body. Some of the scatter radiation enters into areas of the patient's body that are not under examination. Accordingly, these areas of the patient's body not under examination also are exposed to and receive radiation due to the scatter radiation, which needlessly increases the patient's overall exposure to radiation (i.e., the exit radiation dose) and also increases the amount radiation exiting the patient (i.e., the exit radiation dose), which affects the practitioners.

The practitioners are also exposed to the scatter radiation, both the scatter radiation that has not entered the patient's body and the scatter radiation that has entered and exited the patient's body. The scatter radiation from areas of the patient's body that are not under examination, in particular, needlessly increases the amount of radiation that the practitioners are exposed to.

In order to reduce the amount of radiation that the practitioners are exposed to (specifically due to the radiation exiting the patient), lead skirts that are attached to the side of the x-ray table, mobile shields, suspended plexiglass shields, and sterile pads placed on top of or above the patient may be used. Most of these devices are on the top of the examining table and are only designed to shield the practitioners from the radiation exiting the patient. These devices do not protect the patient or the practitioners from excessive radiation (e.g., scatter radiation) and needlessly expose them to the scatter radiation.

Therefore, certain procedures, such as cardiac catheterization, expose areas of the patient's body that do not need to be visualized to radiation, which may needlessly increase both the patient's and the practitioner's overall radiation exposure.

### SUMMARY

In one embodiment, a radiation shield assembly includes a tray having a base portion configured to be positioned underneath at least a portion of a patient, a first side coupled to the base portion at a first non-zero angle, a second side positioned opposite the first side and coupled to the base portion at a second non-zero angle, and a cranial side coupled to the base portion at a third non-zero angle. The radiation shield assembly further includes a first radiation attenuating material that shields radiation, and a first connector for removably coupling the first radiation attenuating material to the tray such that the first radiation material extends downward from the first side, the second side, and the cranial side. The radiation shield assembly also includes a second radiation attenuating material that shields radiation, and a drop-down connector for removably coupling the second radiation attenuating material to the tray such that the second radiation attenuating material extends downward from a side of the tray that is opposite the cranial side and can extend below the table.

In another embodiment a tray for a radiation shield assembly includes a base portion configured to be positioned underneath at least a portion of a patient, a first side coupled to the base portion at a first non-zero angle, a second side positioned opposite the first side and coupled to the base portion at a second non-zero angle, and a cranial side coupled to the base portion at a third non-zero angle. Each of the first side, the second side, and the cranial side define a plurality of openings for securing a radiation attenuating material to the tray.

### BRIEF DESCRIPTION OF THE DRAWINGS

The disclosure will become more fully understood from the following detailed description, taken in conjunction with the accompanying Figures, wherein like reference numerals refer to like elements unless otherwise indicated, in which:

FIG. 1 is a top, left, front perspective view of a radiation shield assembly according to one embodiment;

FIG. 2 is a top, left, front perspective view of the radiation shield assembly of FIG. 1;

FIG. 3 is a top, left, front perspective view of a tray for the radiation shield assembly of FIG. 1;

FIG. 4 is a top view of the tray of FIG. 3;

FIG. 5 is a cross-section view of the tray taken along line V-V of FIG. 4;

FIG. 6 is a side view of the tray of FIG. 3;

FIG. 7a is a front view of a first connector member for a radiation shield assembly according to one embodiment;

FIG. 7b is a side view of a first connector member for a radiation shield assembly according to one embodiment;

FIG. 8 is a top perspective view of a radiation shield assembly according to an embodiment;

FIG. 9 is a top, left, front perspective view of a radiation shield assembly according to an embodiment;

FIG. 10 is a top, left, front perspective view of the radiation shield assembly of FIG. 9;

FIG. 11 is a bottom, right, front perspective view of a radiation shield assembly according to another embodiment;

FIG. 12 is a top, right, front perspective view of the radiation shield assembly of FIG. 11;

FIG. 13 is a bottom, left, front perspective view of the radiation shield assembly of FIG. 11;

FIG. 14 is a top, left, front perspective view of the radiation shield assembly of FIG. 11;

FIG. 15 is a top, right, front perspective view of the radiation shield assembly according to another embodiment; and

FIG. 16 is a top, right, front perspective view of the radiation shield assembly according to another embodiment.

FIG. 17 is a top, right, front perspective view of the radiation shield assembly according to another embodiment.

FIG. 18 is a top, left, front perspective view of a tray for the radiation shield assembly of FIG. 1.

FIG. 19 is a side view of the tray of FIG. 18.

It will be recognized that the Figures are schematic representations for purposes of illustration. The Figures are provided for the purpose of illustrating one or more implementations with the explicit understanding that the Figures will not be used to limit the scope or the meaning of the claims.

### DETAILED DESCRIPTION

Following below are more detailed descriptions of various concepts related to, and implementations of, methods, apparatuses, and for providing a radiation shield assembly and a tray therefor. The various concepts introduced above and discussed in greater detail below may be implemented in any of a number of ways, as the described concepts are not limited to any particular manner of implementation. Examples of specific implementations and applications are provided primarily for illustrative purposes.

### I. Overview

Radiation, specifically, scatter radiation, exposes both the patient and healthcare providers within a healthcare setting to unnecessary radiation. Overtime, exposure to radiation can cause health related issues. Therefore, proper shielding is needed to decrease scatter radiation from reaching healthcare providers while not inhibiting their ability to perform a procedure.

Implementations herein are related to a tray for securing a radiation attenuating material around a radiation source in a healthcare setting.

### II. Overview of Radiation Shield Assembly

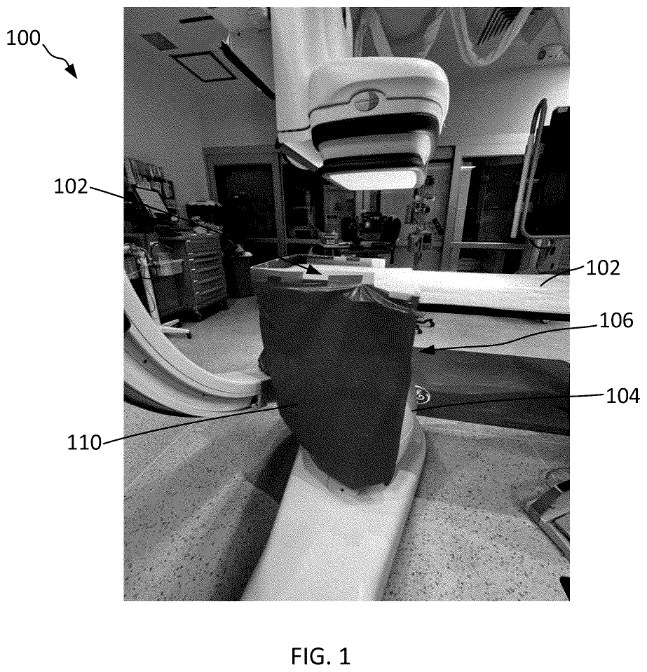

FIG. 1 depicts a radiation shield assembly 100 according to one embodiment. The radiation shield assembly 100 is configured to be used in a hospital setting, such as an operating room. The radiation shield assembly 100 is configured to decrease unwanted radiation from reaching the patient or other people (e.g., physicians, nurses, etc.).

The radiation shield assembly 100 can be disposed on or coupled to a support that positions the radiation shield assembly 100 relative to a radiation source 104. The support can be, for example, a table 102. The table 102 may be, for example, a procedure table, a radiology table, or an examination table. The table 102 is configured to support a patient during a medical procedure. For example, the patient may be laying down on the table 102.

The radiation source 104 is positioned underneath the table 102. However, in other embodiments, the radiation source may be positioned elsewhere in the procedure room. The radiation source 104 is configured to emit radiation 106 (e.g., x-rays, etc.). The radiation source 104 emits x-rays through the table 102 to reach a targeting portion of the patient.

However, during a radiologic procedure, radiation is also deflected outside of the direct radiation beam. Deflected radiation, or scatter radiation, can reach various portions of the patient and others in the room during the procedure. For example, scatter radiation may expose sensitive areas of the patient or the physician to unnecessary radiation.

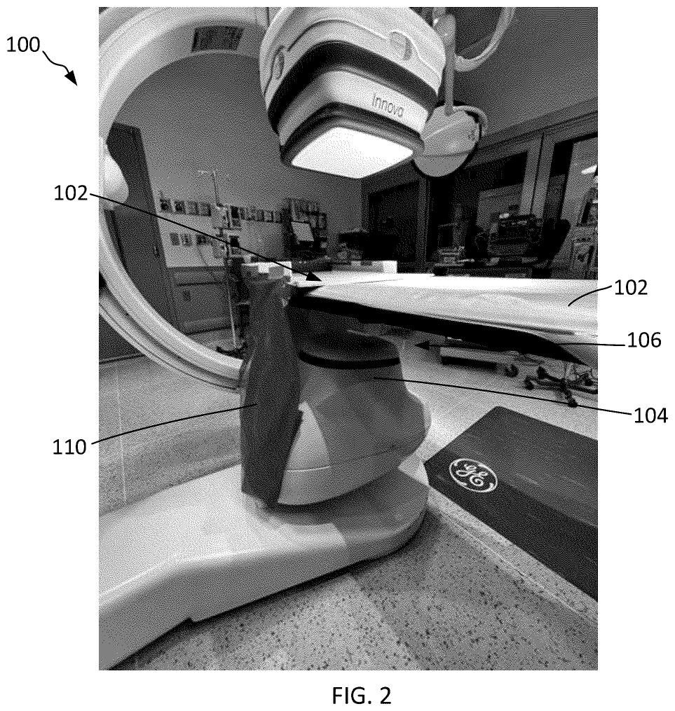

As shown in FIGS. 1-3, the radiation shield assembly 100 includes a tray 108, a radiation attenuating material or shield 110 that shields radiation, and a first connector for connecting the radiation attenuating material 110 to the tray 108.

The tray 108 is positioned near a first end of the table 102. For example, the first end of the table 102 preferably is where the patient's head is positioned. The tray 108 is supported by the table 102. For example, at least a portion (e.g., 40-90%, over 50%, etc.) of the tray 108 is positioned on top of the table 102. The tray 108 receives and supports a portion of the patient. For example, the tray 108 is positioned underneath the patient's head and neck region. The tray 108 comprises (e.g., is composed of, made of, etc.) a material that provides a resilient barrier to, and that will not be denatured by, EPA-registered hospital disinfectants. Further, the tray 108 is radio translucent, such that that radiation, such as x-rays, can pass through the tray 108 and reach a desired portion of the patient.

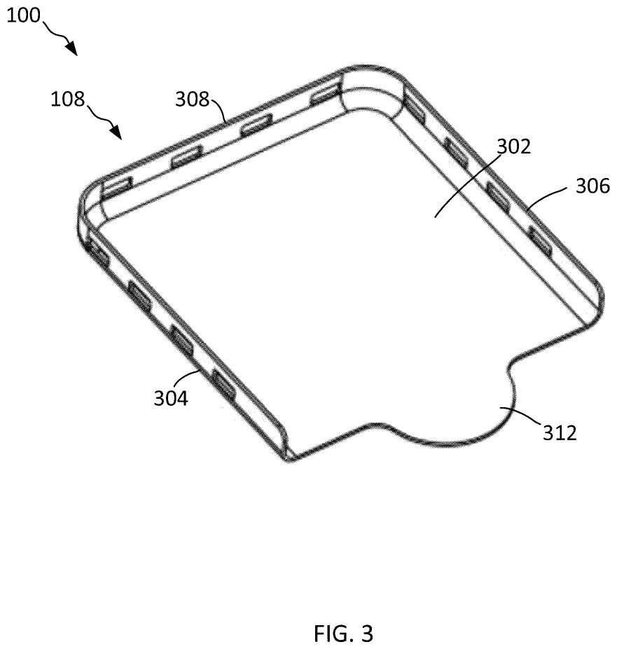

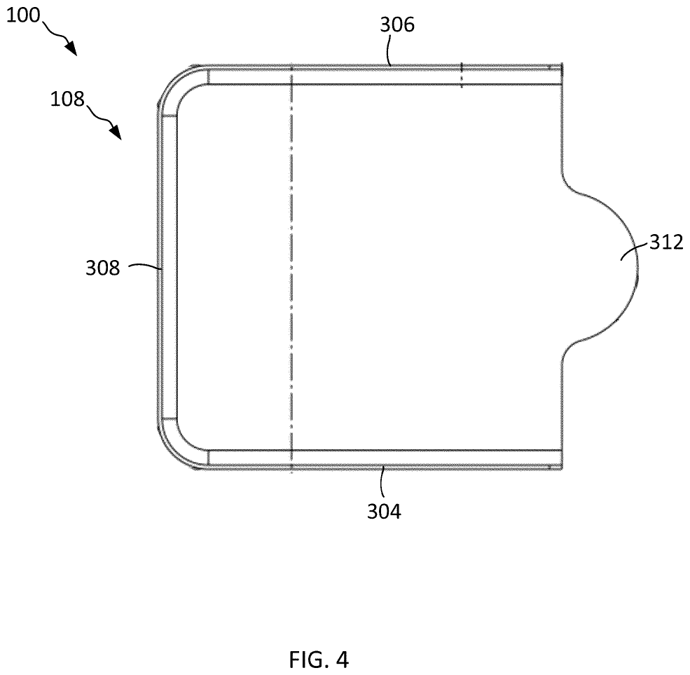

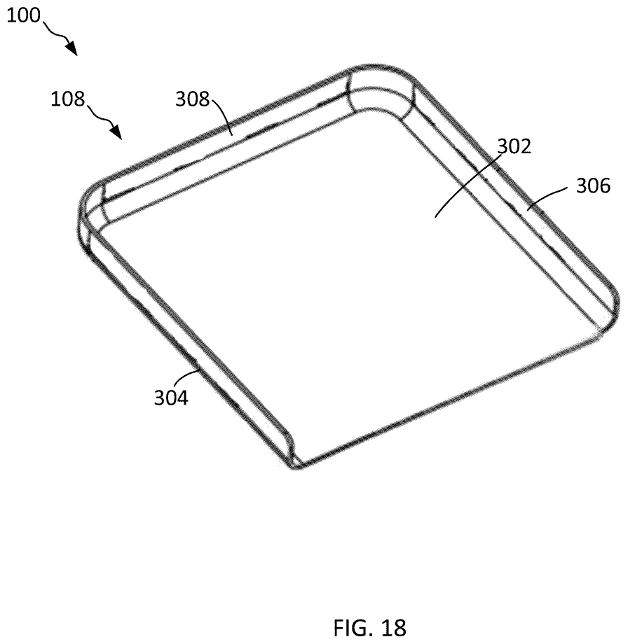

FIGS. 3-6 and 18-19 illustrate the tray 108. FIG. 3 illustrates a perspective view of the tray 108 according to one embodiment. The tray 108 includes a base portion 302, which preferably is in the form of a plate. The base portion 302 is positioned on top of the table 102 and above the radiation source 104 such that at least a portion of the base portion 302 is in contact with the table 102. Preferably the base portion 302 is configured to be positioned between at least a portion of the patient and the radiation source 104. The base portion 302 is configured to allow radiation to flow through the tray 108 and through the patient's body while positioning the radiation attenuating material 110 (e.g., curtain, etc.) to decrease the flow of radiation outward in a direction away from the patient. For example, the base portion 302 is composed of a radio translucent material, such as carbon fiber, but other radio translucent materials are possible.

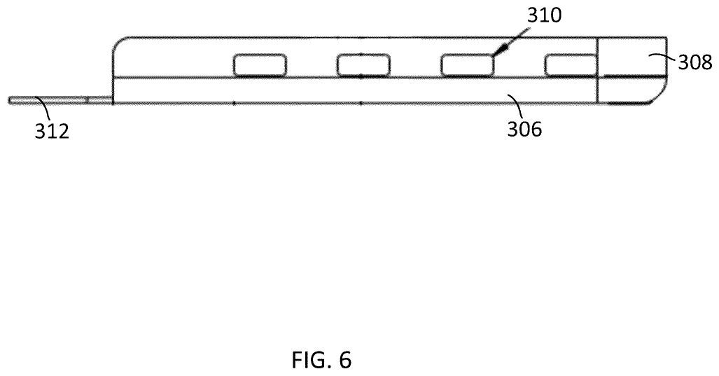

The tray 108 further includes a first side 304. The first side 304 is preferably straight, but could have a curvature or multiple curvatures. As shown in FIG. 6, the first side 304 is coupled to, and preferably contiguous with, the base portion 302. The first side 304 and the base portion 302 can be coupled by being a continuous and unitary structure or the first side 304 and the base portion 302 can be separate structures that are coupled. The first side 304 is positioned at a non-zero angle (e.g., between 45 degrees to 90 degrees, etc.) relative to the base portion 302. The first side 304 extends a distance above the base portion 302. For example, the first side may extend a few inches (e.g., 2 inches, 4 inches 6 inches, etc.) above the base portion 302.

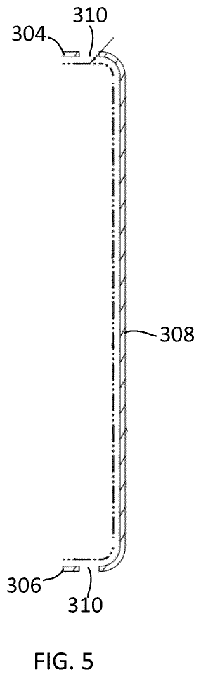

The tray 108 also includes a second side 306. The second side 306 is preferably straight, but could have a curvature or multiple curvatures. As shown in FIG. 5, the second side 306 is coupled to, and preferably contiguous with, the base portion 302. The second side 306 and the base portion 302 can be coupled by being a continuous and unitary structure or the second side 306 and the base portion 302 can be separate structures that are coupled. The second side 306 is positioned at a non-zero angle (e.g., between 45 degrees to 90 degrees, etc.) relative to the base portion 302. The second side 306 extends a distance above the base portion 302. For example, the first side may extend a few inches (e.g., 2 inches, 4 inches 6 inches, etc.) above the base portion 302.

The tray 108 also includes a cranial side 308. The cranial side 308 is preferably straight, but could have a curvature or multiple curvatures. The cranial side 308 is coupled to, and preferably contiguous with, each of the base portion 302, the first side 304, and the second side 306. The cranial side 308, the base portion 302, the first side 304, and the second side 306 can be coupled by being a continuous and unitary structure or the cranial side 308, the base portion 302, the first side 304, and the second side 306 can be separate structures that are coupled. The cranial side 308 is positioned at a non-zero angle (e.g., between 45 degrees to 90 degrees, etc.) relative to the base portion 302 and substantially perpendicular to each of the first side 304 and the second side 306. For example, the first side 304 is parallel to the second side 306 and the cranial side is perpendicular to each of the first side 304 and the second side 306. The cranial side 308 is positioned above a top of the patient's head, while each of the first side 304 and the second side 306 are adjacent to a left and right side of the patient, respectively. The cranial side 308 extends a distance above the base portion 302. For example, the cranial side 308 may extend a few inches (e.g., 2 inches, 4 inches 6 inches, etc.) above the base portion 302.

According to this embodiment, each of the first side 304, the second side 306, and the cranial side 308 extend a distance (e.g., a few inches, 2 inches, 4 inches, etc.) above the base portion 302. For example, each of the first side 304, the second side 306, and the cranial side 308 extend about 2 inches above the base portion 302 to allow the physician to access the patient's head, neck, and thoracic regions. For example, the tray 108 allows the physician unrestricted access to the patient's head, neck, and thoracic regions during medical procedures. In other embodiments, each of the first side 304, the second side 306, and the cranial side 308 may extend a distance less than or greater than 2 inches.

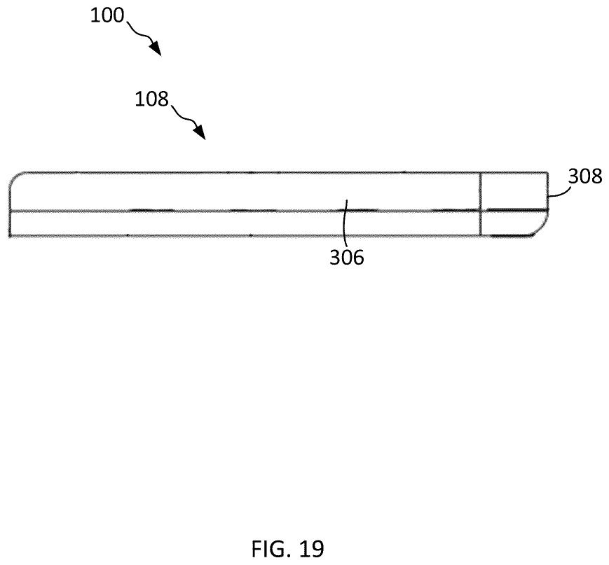

As shown in FIG. 3, the tray 108 includes a projection or tab 312. The projection 312 is positioned on an end of the base portion 302 opposite the cranial side 308 (e.g., on a caudal side of the base portion 302). The projection 312 preferably is substantially semi-circular in shape. The projection 312 preferably is configured to be slid underneath the patient and may assist in maintaining the base portion 302 of the tray 108 in position between the patient and the table 102. The projection 312 is also configured to be flat such that the projection is substantially flush with the table 102. As shown in FIG. 19, in some embodiments, the tray 108 does not include the projection 312, and instead includes a straight (e.g., flat, etc.) edge.

The radiation shield assembly 100 further includes the radiation attenuating material 110 for attenuating radiation. The radiation attenuating material 110 comprises, for example, material such as lead or the like that attenuates or decreases radiation, such as x-rays, from flowing through the radiation attenuating material 110. The radiation attenuating material 110 is coupled to the tray 108. The radiation attenuating material is positioned below the table 102 and surrounds at least a portion of the radiation source 104. The radiation attenuating material 110 preferably is flexible and moveable. For example, the radiation attenuating material 110 is curtain-like such that a portion of the radiation attenuating material 110 may be moved or displaced by the user. The radiation attenuating material 110 preferably is a single continuous curtain of material, but the radiation attenuating material 110 may be divided into separate segments, e.g., a segment for each side of the tray 108.

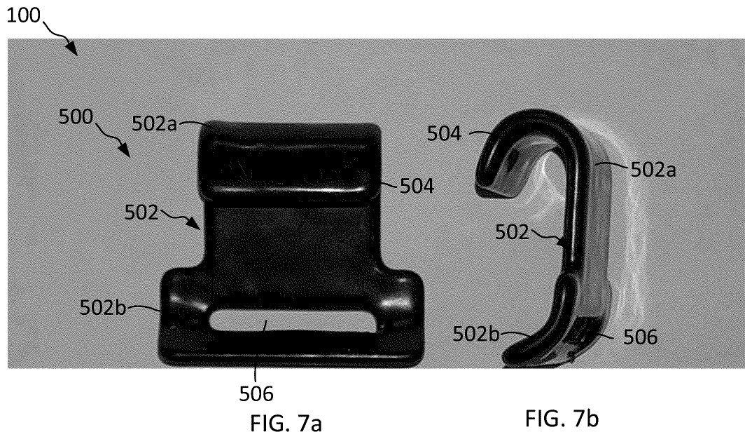

The radiation shield assembly 100 further includes the first connector for connecting the radiation attenuating material 110 to the tray 108, and preferably to the first side 304, the second side 306, and the cranial side 308 of the tray 108. As shown in FIGS. 7a-7b, the first connector can include a plurality of first connector members 500 (e.g., fasteners, etc.). The plurality of first connector members 500 may be substantially hook shaped. The plurality of first connector members 500 are configured to couple an object (e.g., a radiation curtain, a shield, a first radiation attenuating material, etc.) to the tray 108. For example, the first connector members 500 are configured to removably or releasably couple the radiation attenuating material 110 to the tray 108.

Each of the first connector members 500 preferably include a body 502, and a hook 504. The body 502 includes a first end 502a and a second end 502b. The second end 502b is positioned opposite the first end 502a. The second end 502b may be curved and configured to pivot (e.g., rotate, swivel, etc.) above the body 502. The first end 502a is coupled to the hook 504. The hook 504 is selectively coupled to at least one of the first side 304, the second side 306, or the cranial side 308. For example, the hook 504 is positioned on an edge of the tray 108, preferably, on an upper edge of one of the first side 304, the second side 306, or the cranial side 308, to selectively couple the first connector member 500 to the tray 108. As an alternative configuration, the tray 108 can include a plurality of openings 310 and the hook 504 can be positioned within a respective one of the plurality of openings 310 on one of the first side 304, the second side 306, or the cranial side 308, to selectively couple each of the first connector members 500 to the tray 108.

Further, the second end 502b of the body 502 includes a slot 506. The slot 506 can be substantially rectangular in shape. The slot 506 allows an object, such as a radiation attenuating material 110 to be coupled to the first connector member 500 such that the first connector can selectively couple the radiation attenuating material 110 to the tray 108. For example, the radiation attenuating material 110 may be secured to the first connector member 500 via a clip, Velcro, or any other means of securement (e.g., connected through an aperture of the radiation attenuating material 110, etc.). According to some embodiments, the second end 502b includes at least one of a clamp or an aperture configured to secure a portion of the radiation shield attenuating material 110 to the first connector member 500.

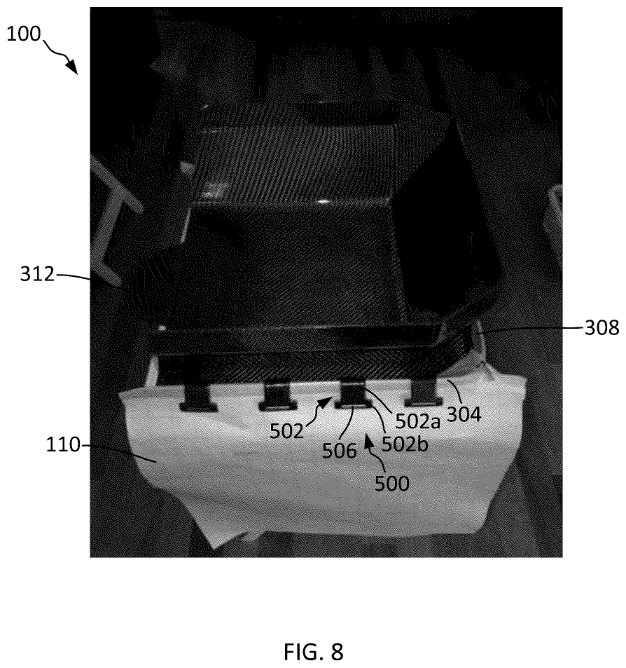

As shown in FIG. 8, the plurality of first connector members 500 can removably or releasably couple the radiation attenuating material 110 to the tray 108. For example, the first connectors 500 may be placed (e.g., hook onto, etc.) on the tray 108, preferably to the first side 304, the second side 306, and the cranial side 308.

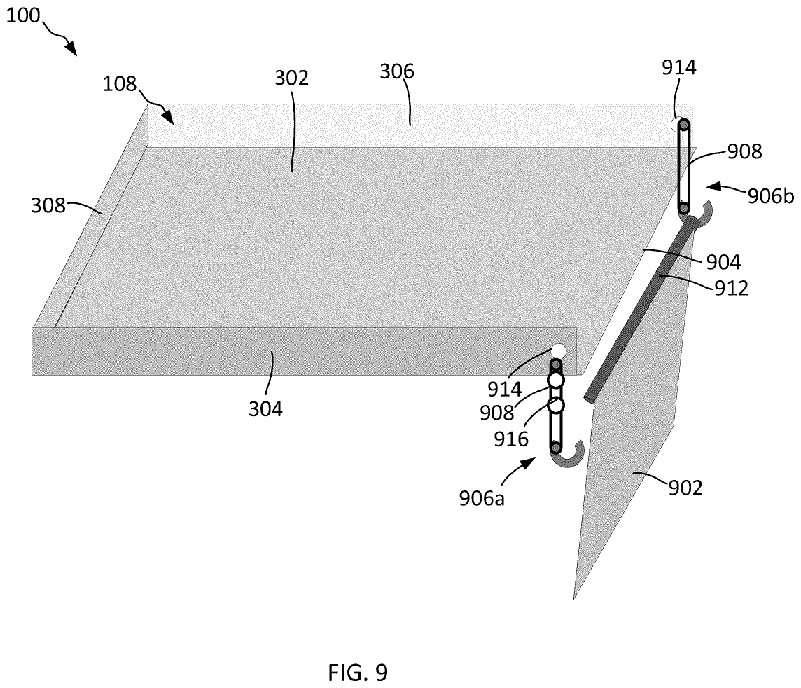

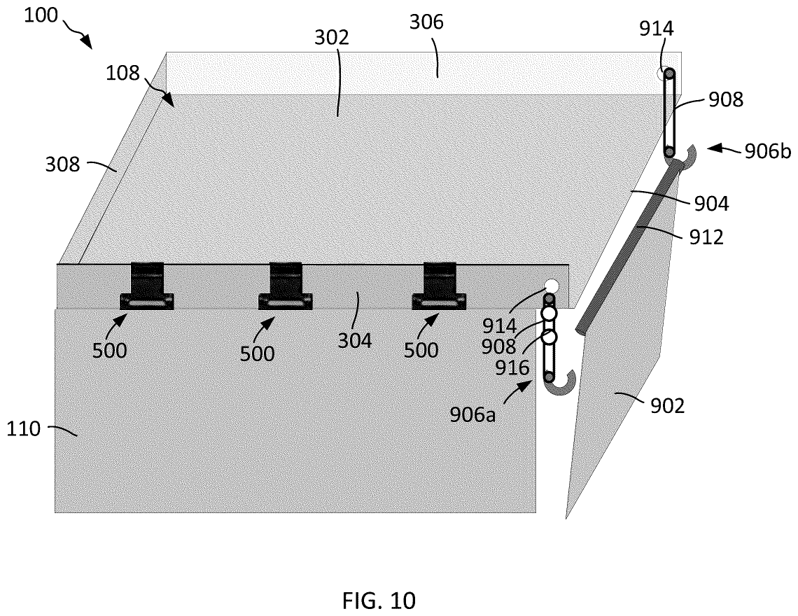

FIGS. 9-10 illustrate the radiation shield assembly 100 including the tray 108, and a second radiation attenuating material (e.g., a second shield) 902. According to this embodiment, the second radiation attenuating material 902 is positioned below the table 102. The second radiation attenuating material 902 is positioned to be between at least a portion or side of the radiation source 104 and a healthcare worker to decrease the flow of radiation outward in a direction away from the patient. Together, the radiation attenuating material 110 and the second radiation attenuating material 902 may be positioned around all sides of the radiation source 104 (e.g., 360 degrees around the radiation source 104) such that the flow of radiation outward (e.g., along a plane parallel to the table 102, etc.) away from the patient is decreased.

Similar to the radiation attenuating material 110, the second radiation attenuating material 902, preferably, is flexible and moveable. For example, the second radiation attenuating material 902 is also curtain-like such that a portion of the second radiation attenuating material 902 may be moved or displaced by the user. The second radiation attenuating material 902 preferably is a single continuous curtain of material, but the second radiation attenuating material 902 also may be divided into separate segments.

As shown in FIG. 10, the radiation attenuating material 110 extends downward from (e.g., hangs from, etc.) the tray, preferably, from the first side 304, the second side 306, and the cranial side 308 of the tray 108, while the second radiation attenuating material extends downward from an end 904 of the tray 108 opposite the cranial side 308. For example, the radiation attenuating material 110 and the second radiation attenuating material 902 preferably surround (e.g., enclose, encapsulate, etc.) the radiation source 104. By surrounding the radiation source on multiple sides (e.g., four sides, the first side 304, the second side 306, the cranial side 308, and the end 904, etc.), the radiation attenuating material 110 and the second radiation attenuating material 902 decrease (e.g., reduce, etc.) the flow of radiation outward in a direction away from the patient. For example, the radiation attenuating material 110 and the second radiation attenuating material 902 decrease or reduce the amount of radiation that reaches healthcare workers in the room and the amount of radiation that is received by the patient's body positioned off of the tray 108 on the table (e.g., mid back region, legs, feet, etc.).

As shown in FIG. 10, the radiation attenuating material 110 and the second radiation attenuating material 902 are positioned a distance away from the radiation source 104 such that the radiation attenuating material 110 and the second radiation attenuating material 902 surround the radiation source 104 such that flow of radiation scattered outward from the patient from colliding with a bottom side of the table 102 is decreased. Decreasing the flow of radiation outward away from the patient decreases the amount of radiation contacting healthcare workers in the procedure room.

As shown in FIG. 10, the first connector member 500 can be a hook 504 is positioned on an edge of the tray 108, preferably, on an upper edge of one of the first side 304, the second side 306, or the cranial side 308, to selectively couple the first connector member 500 to the tray 108. As an alternative configuration, openings (not shown) could be provided in the tray 108, and the hook 504 can be positioned within a respective one of a plurality of openings on one of the first side 304, the second side 306, or the cranial side 308, to selectively couple each of the first connector members 500 to the tray 108.

Further, as shown in FIG. 9-10, the radiation shield assembly 100 further includes a second or drop-down connector for connecting the second radiation attenuating material 902 to the tray 108. Preferably the drop-down connector releasably connects the radiation attenuating material 902 to the tray 108 such that the radiation attenuating material 902 extends below the table 102. Preferably the drop-down connector is configured to adjustably connect the second radiation attenuating material 902 to the tray 108, e.g., such that a position of the second radiation attenuating material 902 can be adjusted (e.g., spatially, orientationally, etc.) relative to the tray 108. For example, a distance and/or orientation between the second radiation attenuating material 902 and the tray can be adjusted. The drop-down connector can include at least one drop-down connector member in the form of drop-down hook 906. For example, the radiation shield assembly 100 preferably includes a first drop-down connector member in the form of a drop-down hook 906a and a second drop-down connector member in the form of a drop-down hook 906b. The first drop-down hook 906a is coupled to the first side 304 of the tray 108 and the second drop-down hook 906b is coupled to the second side 306 of the tray 108 opposite the first side 304. The drop-down hooks 906 extend a distance below the tray 108 and are configured to support the second radiation attenuating material 902.

The drop-down hooks 906 include a body 908 and a curved end 910. The body 908 is coupled to the tray 108 and the curved end receives the second radiation attenuating material 902. For example, the curved end 910 is hook shaped such that the second radiation attenuating material 902 can quickly be hung and removed from the curved ends 910.

As shown in FIG. 9-10 the second radiation attenuating material 902 includes a rod 912. The rod 912 is coupled to the second radiation attenuating material 902. For example, the second radiation attenuating material 902 may include a loop (e.g., a sewn loop on an end, etc.) that the rod 912 is positioned within. The rod 912 is positioned adjacent to the projection 312 of the tray 108. For example, the rod 912 is positioned below the projection 312 and is removably or releasably coupled to the drop-down hooks 906.

In other embodiments, the rod 912 may be clamped onto the second radiation attenuating material 902. The rod 912 is received by the drop-down hooks 906 and supports the second radiation attenuating material 902. For example, the rod 912 may be a lightweight material (e.g., aluminum, etc.) that provides structural support to the second radiation attenuating material 902 such that the second radiation attenuating material 110 does not sag or fall down while hanging from the drop-down hooks 906.

In other embodiments, the second radiation attenuating material 902 may be hooked onto the rod 912. For example, the second radiation attenuating material 902 may be coupled to a plurality of hooks that are hung from the rod 912.

In other embodiments, the radiation shield assembly 100 does not include the rod 912. Instead, the second radiation attenuating material 902 may include a rigid end (e.g., rigid edge, etc.) with slots that receive the drop-down hooks 906 to hang the second radiation attenuating material 902.

The drop-down hooks 906 may be coupled to the tray 108 using any suitable coupling mechanism. For example, the first side 304 and the second side 306 may each include an opening 914 (e.g., a hole, a drilling, etc.) configured to receive a bolt coupled to the drop-down hooks 906. A nut, or a washer, may be placed on either side of each of the first side 304 and the second side 306 to secure the bolts within the openings.

The drop-down hooks 906 may include joints or a plurality of pivot points 916 such that the drop-down hooks 906 may be moved (e.g., bend, flex, rotate, etc.) to accommodate orientational changes between the second radiation attenuating material 902 and the tray 109, for example to accommodate various equipment (e.g., the radiation source, etc.) positioned underneath the tray 109. Additionally or alternatively, the drop-down connector may include conventional means that permit adjustment of the spatial orientation of the second radiation attenuating material 902 to the tray 108 (e.g., adjusting the space between the second radiation attenuating material 902 and the tray 108) to accommodate adjustability and configurability for different associated equipment.

As shown in FIG. 10, the second radiation attenuating material 902 is not connected to the first radiation attenuating material 110. For example, the second radiation attenuating material 902 is coupled to the tray 108 (e.g., via the rod 912, etc.) such that the second radiation attenuating material 902 can move independently with respect to the first radiation attenuating material 110. Additionally or alternatively, the second radiation attenuating material 902 may be coupled to the first radiation attenuating material 110. For example, the second radiation attenuating material 902 may be selectively coupled to the first radiation attenuating material 110. For example, the first radiation attenuating material 110 may be coupled to (via connectors 500, etc.) to the tray 108, and then the second radiation attenuating material 902 may be coupled (e.g., via the drop-down hooks 906, etc.) to the tray 108 and also to the first radiation attenuating material 110 via any suitable coupling mechanism to provide 360 degree shielding around the radiation source 104 to reduce the amount of radiation that the practitioners are exposed to. The coupling mechanism between the first radiation attenuating material 110 and the second radiation attenuating material 902 may be a zipper, Velcro, snaps, but is not limited thereto.

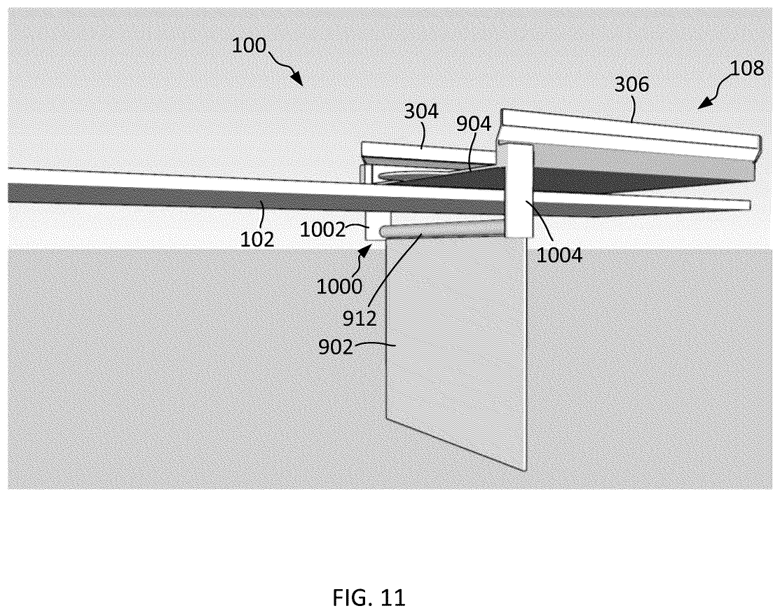

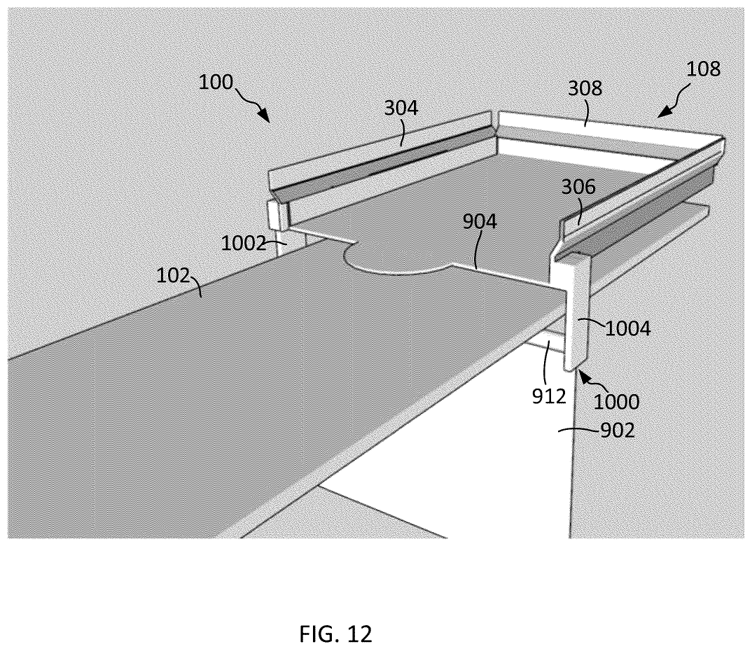

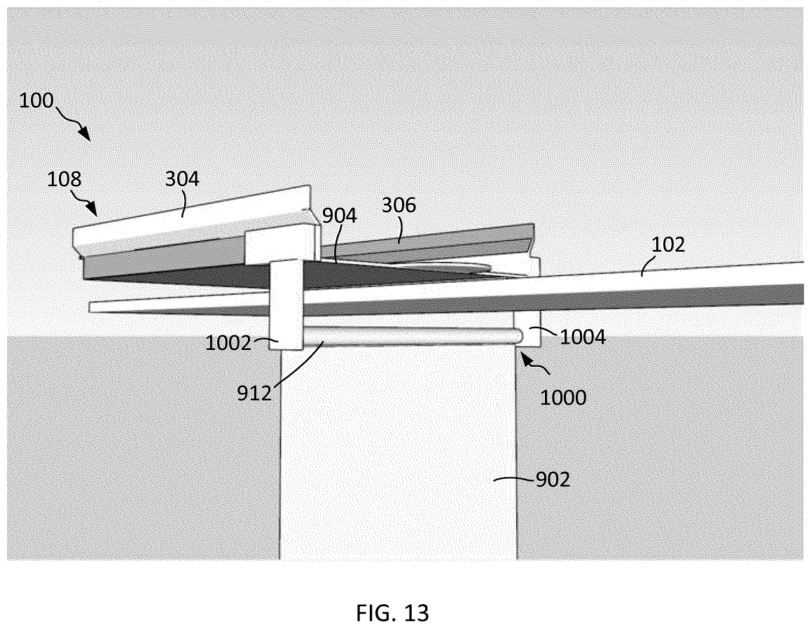

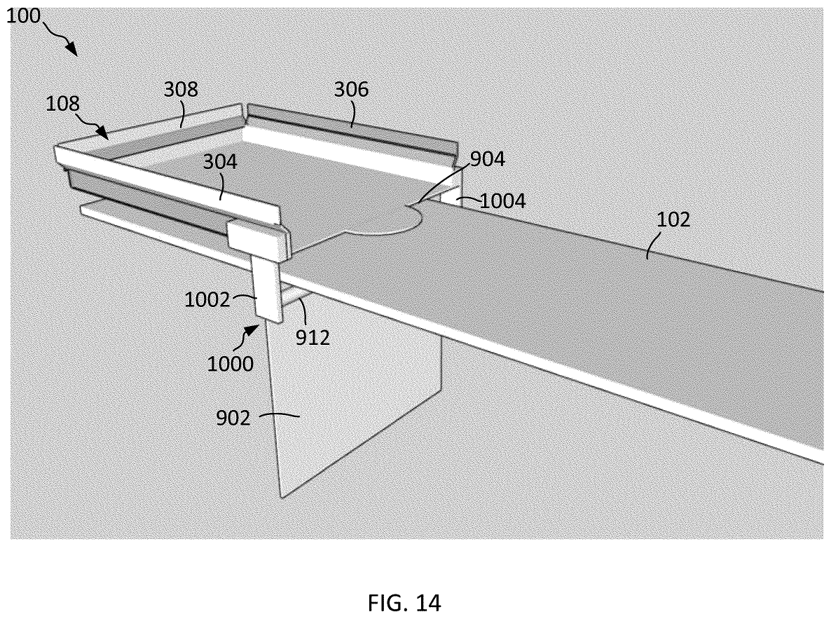

FIGS. 11-16 illustrate the radiation shield assembly 100 including the tray 108, the second radiation attenuating material (e.g., a second shield) 902, and a second or drop-down connector 1000 according to another embodiment. Similar to the embodiment of FIGS. 9-10, the second radiation attenuating material is positioned below the end 904 of the tray 108 opposite the cranial side 308. As shown in FIGS. 11-14, the drop-down connector 1000 includes a first drop-down connector member 1002 and a second drop-down connector member 1004. The first drop-down connector member 1002 is coupled to the first side 304 of the tray 108 and the second drop-down connector member 1004 is coupled to the second side 306 and positioned opposite the first drop-down connector member 1002. In some embodiments, the first drop-down connector member 1002 and the second drop-down connector member 1004 may be coupled to the tray 108 with a securing mechanism (e.g., a screw, a bolt, etc.) In other embodiments, the first drop-down connector member 1002 and the second drop-down connector member 1004 may be integrally formed with the tray 108.

Each of the first drop-down connector member 1002 and the second drop-down connector member 1004 may include a protrusion (e.g., a first protrusion, etc.). The protrusions may be positioned on an end of the first drop-down connector member 1002 and the second drop-down connector member 1004 opposite the tray 108. The protrusion is configured to engage with the rod 912. For example, the rod 912 may be adjustable such that the rod 912 can contract and expand to secure onto the protrusions. For example, the rod 912 may be a telescoping rod or a flexible rod that can easily deform to engage with the protrusions and secure the second radiation attenuating material 902 to the tray 108. In other embodiments, the first drop-down connector member 1002 and the second drop-down connector member 1004 may include hooks as previously described in FIGS. 9-10. For example, the hooks may be integrally form with each of the first drop-down connector member 1002 and the second drop-down connector member 1004 or attached via a securing mechanism (e.g., a screw, a bolt, etc.) to each of the first drop-down connector member 1002 and the second drop-down connector member 1004.

As shown in FIGS. 11-14, the first drop-down connector member 1002 and the second drop-down connector member 1004 extend a distance below the tray 108. For example, the first drop-down connector member 1002 and the second drop-down connector member 1004 may extend a few inches (e.g., between 2 inches and 8 inches, between 4 inches and 6 inches, etc.) below the tray 108 such that first drop-down connector member 1002 and the second drop-down connector member 1004 extend below the table 102. Additionally or alternatively, the drop-down connector 1000 may include conventional means that permit adjustment of a position of the second radiation attenuating material 902 relative to the tray 108 (e.g., adjusting the space between the second radiation attenuating material 902 and the tray 108) to accommodate adjustability and configurability for different associated equipment. For example, the length of the first and second drop-down connector member 1002 and 1004 may be adjustable such that the second radiation attenuating material 902 can be moved closer or further away from the table 102 or be adjusted to accommodate various dimensions of tables 102.

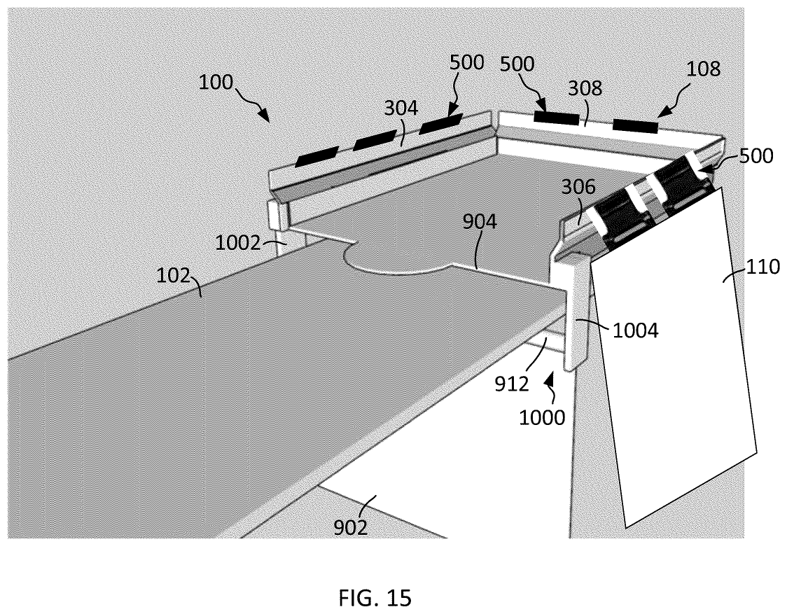

Now referring to FIG. 15, the radiation shield assembly 100 includes the tray 108, the first radiation attenuating material 110 (e.g., the first shield, the plurality of first shields, etc.), the second radiation attenuating material (e.g., a second shield) 902, and the drop-down connector 1000. According to this embodiment, the first radiation attenuating material 110 may be a single continuous curtain of material, or may be divided into separate segments, e.g., a segment for each side of the tray 108. For example, the first radiation attenuating material 110 is coupled to each side of the tray by first connectors 500 regardless as to if the first radiation attenuating material 110 is a single continuous curtain of material, or is divided into separate segments. As shown in FIG. 15, the radiation attenuating material 110 and the second radiation attenuating material 902 are positioned such that a radiation source may be shielded (e.g., surrounded by radiation attenuating material on all sides, etc.) on all sides (e.g., 360 degree shielding, etc.). Additionally or alternatively, the first radiation attenuating material 110 and the second radiation attenuating material 902 can be a single continuous member forming a singular radiation attenuating material (e.g., shield, etc.) that forms a box or an enclosure around the radiation source 104.

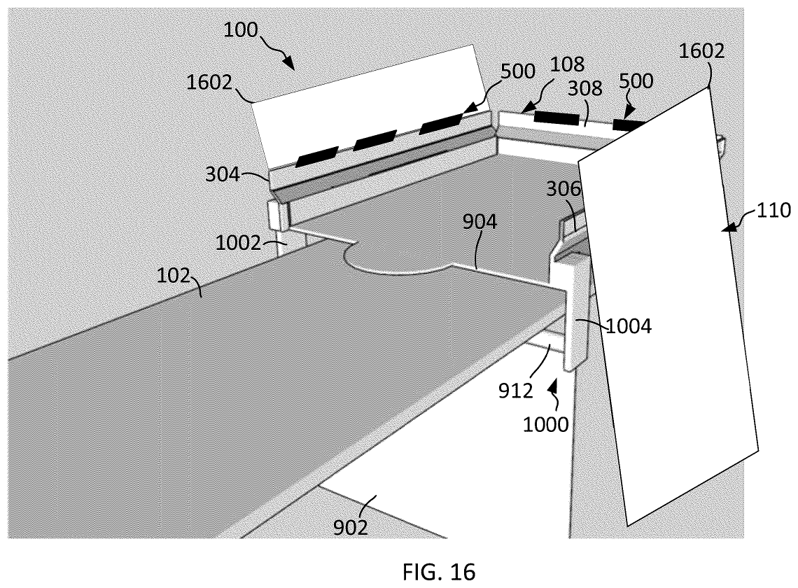

FIG. 16 illustrates another embodiment of the radiation shield assembly 100 that is similar to the embodiment shown in FIG. 15. As shown in FIG. 16 the radiation attenuating material 110 includes an extension portion (e.g., extension shield, an extension radiation attenuating material, etc.) 1602 that extends above the tray 108. The extension portion 1602 extends the radiation attenuating material 110 in an upward direction (e.g., in the direction of the first side 304, the cranial side 308 and the second side 306, etc.) from the tray 108. The extension portion 1602 can be continuous with the remainder of the radiation attenuating material 110 (below the tray 108), such that according to the embodiment of FIG. 16 the radiation attenuating material 110 is longer than in other embodiments. Alternatively, the extension portion 1602 can be a separate, additional portion of the radiation attenuating material 110 that is separate from the portion of the radiation attenuating material 110 that is below the tray 108. In another alternative embodiment, the extension portion 1602 can be continuous with the remainder of the radiation attenuating material 110 and can also have varying lengths such that the height of the extension portion 1602 is adjustable. For example, the radiation attenuating material 110 may be longer (e.g., an extending radiation attenuating material 110, etc.) such that the height of the radiation attenuating material 110 extends upward above the tray 108 to a desirable height.

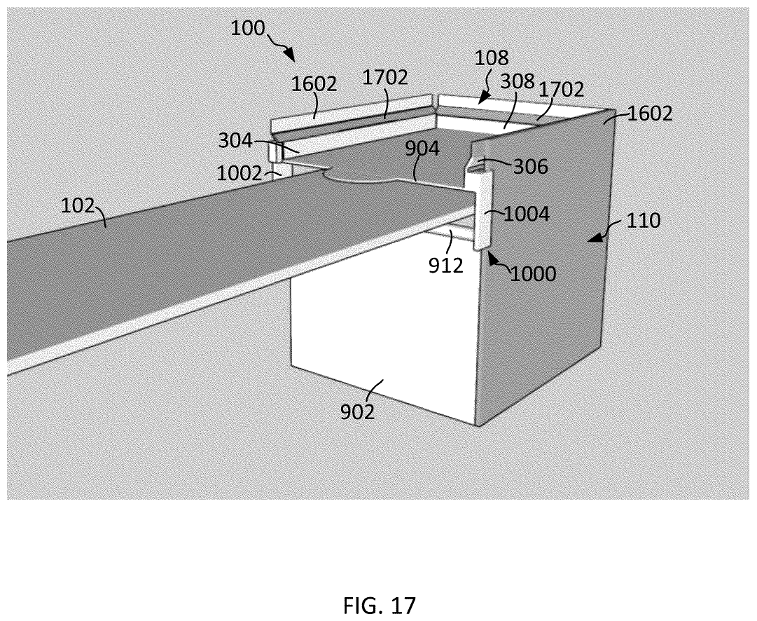

FIG. 17 illustrates another embodiment of the radiation shield assembly 100 that is similar to the embodiments of FIGS. 15 and 16. As shown in FIG. 167, the extension portion 1602 is continuous with the radiation attenuating material 110. According to this embodiment, the radiation attenuating material 110 including the extension portion 1602 can be coupled to the radiation tray 108 by any suitable connector, such as the connectors 500. In an alternative embodiment, the radiation attenuating material 110 including the extension portion 1602 may include an integrated hook 1702 that couples the radiation attenuating material to the sides (e.g., the first side 304, the second side 306, the third side 308, etc.). According to this embodiment, the first radiation attenuating material 110 is continuous with the second radiation attenuating material 902. For example, the first radiation attenuating material 110 and the second radiation attenuating material 902 are one continuous sheet of radiation attenuating material that wraps around the radiation source 104 (e.g., forming a box, forming an enclosure, etc.) such that there are no seams or gaps decreasing the flow of radiation outward underneath the table 102. In an alternative embodiment, the first radiation attenuating material 110 may include a first fastener (e.g., snaps, Velcro, etc.) that can be coupled to a second faster (e.g., complementary snaps, Velcro, etc.) of the second radiation attenuating material 902 to decrease a space between the first radiation attenuating material 110 and the second radiation attenuating material 902, thus decreasing the flow of radiation outward (e.g., toward a healthcare worker, a physician, etc.) underneath the table 102.

The radiation shield assembly 100 may include a plurality of extension portions 1602, such that each of the first side 304, the second side 306, and the cranial side 308 may include an extension portion 1602. The one or more extension portion 1602 may extend a distance above the sides of the tray 108. For example, the one or more extension portion 1602 may extend in a range between 2 inches to 8 inches above sides of the tray 108 but is not limited thereto such that the one or more extension portions 1602 may extend a distance less than 2 inches or greater than 8 inches above the sides of the tray 108. According to some embodiments, when a patient is positioned on top of the tray 108, the shield may extend at least 2 inches above the patient's body (e.g., chest, etc.) such that healthcare workers can still reach the patient's body during the procedure.

The one or more extension portions 1602 are configured to decrease the flow of radiation outward in a direction away from the patient. For example, radiation may be directed towards a portion of the patient's body for imaging purposes (e.g., from below the tray 108), thus the one or more extension portions 1602 may decrease the flow of radiation outward in a direction away from the patient above the table 102. The first radiation attenuating material 110, including the one or more extension portions 1602, and the second radiation attenuating material 902 may decrease the flow of radiation outward in a direction away from the patient both below and above the table 102.

In some embodiments, the radiation shield assembly 100 may include two extension portions 1602. A first extension portion 1602 may be coupled to the first side 304 and a second extension portion 1602 may be coupled to the second side 306, leaving the cranial side 308 open. In other embodiments, the radiation shield assembly 100 includes three extension portions on each of the first side 304, the second side 306, and the cranial side 308.

In some embodiments, the one or more extension portions 1602 may extend in a direction transverse to (e.g., perpendicular to, etc.) the table 102 (e.g., parallel to the first side 304, the second side 306, and the cranial side 308, etc.). In other embodiments, the one or more extension portions 1602 may extend at an angle relative to the sides of the tray 108. As shown in FIG. 16, the first connectors 500 are not visible from an exterior side of the tray 108. The first connectors 500 are coupled to an interior side (e.g., a side closest to the patient, etc.) of the radiation attenuating material 110. For example, a pocket or a strap may be positioned around the first connectors 500 coupling the first connectors 500 to the radiation attenuating material 110. The radiation attenuating material 110 including the extension portion 1602 may decrease the flow of radiation outward from the patient.

By coupling the radiation attenuating material 110 directly to the tray 108, gaps between the table 102 and the radiation attenuating material 110 are decreased, thus the flow of radiation outward away from the patient is also decreased. Further, coupling the radiation attenuating material 110 to the tray 108, decreases the extra load (e.g., weight of the radiation attenuating material 110, etc.) on the edges of the table 102 and/or rails coupled to the table.

### II. Configuration of Example Embodiments

As utilized herein, an area is measured along a plane (e.g., a two-dimensional plane, etc.) unless otherwise indicated. This area may change in a direction that is not disposed along the plane (e.g., along a direction that is orthogonal to the plane, etc.) unless otherwise indicated.

While this specification contains many specific implementation details, these should not be construed as limitations on the scope of what may be claimed but rather as descriptions of features specific to particular implementations. Certain features described in this specification in the context of separate implementations can also be implemented in combination in a single implementation. Conversely, various features described in the context of a single implementation can also be implemented in multiple implementations separately or in any suitable subcombination. Moreover, although features may be described as acting in certain combinations and even initially claimed as such, one or more features from a claimed combination can, in some cases, be excised from the combination, and the claimed combination may be directed to a subcombination or variation of a subcombination.

As utilized herein, the terms “substantially,” “generally,” “approximately,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the appended claims.

The term “coupled” and the like, as used herein, mean the joining of two portions or components directly or indirectly to one another. Such joining may be stationary (e.g., permanent) or moveable (e.g., removable or releasable). Such joining may be achieved with the two portions or components or the two portions or components and any additional intermediate portions or components being integrally formed as a single unitary body with one another, with the two portions or components, or with the two portions or components and any additional intermediate portions or components being attached to one another.

It is important to note that the construction and arrangement of the various systems shown in the various example implementations is illustrative only and not restrictive in character. All changes and modifications that come within the spirit and/or scope of the described implementations are desired to be protected. It should be understood that some features may not be necessary, and implementations lacking the various features may be contemplated as within the scope of the disclosure, the scope being defined by the claims that follow. When the language “a portion” is used, the item can include a portion and/or the entire item unless specifically stated to the contrary.

Also, the term “or” is used, in the context of a list of elements, in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

Additionally, the use of ranges of values (e.g., W1 to W2, etc.) herein are inclusive of their maximum values and minimum values (e.g., W1 to W2 includes W1 and includes W2, etc.), unless otherwise indicated. Furthermore, a range of values (e.g., W1 to W2, etc.) does not necessarily require the inclusion of intermediate values within the range of values (e.g., W1 to W2 can include only W1 and W2, etc.), unless otherwise indicated.

What is claimed is:

1. A radiation shield assembly comprising:
a tray configured to be positioned on a table supporting the patient and comprising:
a base portion configured to be positioned underneath at least a portion of a patient,
a first side coupled to the base portion at a first non-zero angle,
a second side positioned opposite the first side and coupled to the base portion at a second non-zero angle, and
a cranial side coupled to the base portion at a third non-zero angle;
a first radiation attenuating material that shields radiation;
a first connector for removably coupling the first radiation attenuating material to the tray such that the first radiation material extends downward from first side, the second side, and the cranial side;
a second radiation attenuating material that shields radiation; and
a drop-down connector for removably coupling the second radiation attenuating material to the tray such that the second radiation attenuating material extends downward from a side of the tray that is opposite the cranial side and can extend below the table.

2. The radiation shield assembly of claim 1, wherein the base portion of the tray is configured to support the head and neck of the patient.

3. The radiation shield assembly of claim 1, wherein the base portion of the tray is radio translucent.

4. The radiation shield assembly of claim 1, wherein the base portion of the tray further comprises a projection positioned on an end of the base portion opposite the cranial side.

5. The radiation shield assembly of claim 1, wherein each of wherein the base portion, the first side, the second side, and the cranial side of the tray are formed of a material that provides a resilient barrier to, and that will not be denatured by, EPA-registered hospital disinfectants.

6. The radiation shield assembly of claim 1, wherein the base portion, the first side, the second side, and the cranial side of the tray comprise carbon fiber.

7. The radiation shield assembly of claim 1, wherein the cranial side is contiguous with each of the first side and the second side.

8. The radiation shield assembly of claim 1, wherein the first non-zero angle, the second non-zero angle, and the third non-zero angle are each between 45 degrees to 90 degrees.

9. The radiation shield assembly of claim 1, wherein the first non-zero angle, the second non-zero angle, and the third non-zero angle are substantially equal.

10. The radiation shield assembly of claim 1, wherein the cranial side is substantially perpendicular to each of the first side and the second side.

11. The radiation shield assembly of claim 1, wherein the first connector comprises a plurality of first connector members.

12. The radiation shield assembly of claim 11, wherein the first connector members are each hook shaped and are hooked onto the first side, the second side, and the cranial side.

13. The radiation shield assembly of claim 11, wherein the first connector members each comprise:
a first end with a hook selectively coupled to at least one of the first side, the second side, and the cranial side; and
a second end coupled to the radiation attenuating material.

14. The radiation shield assembly of claim 13, wherein the second end comprises at least one of a clamp or an aperture configured to secure a portion of the radiation attenuating material.

15. The radiation shield assembly of claim 1, wherein the drop-down connector is adjustable to adjust a position of the second radiation attenuating material relative to the tray.

16. The radiation shield assembly of claim 1, wherein the drop-down connector comprises:
a first drop-down connector member coupled to the first side and extending a first distance below the tray,
a second drop-down connector member coupled to the second side and positioned opposite the first drop-down connector member, the second drop-down connector member extending a second distance below the tray, and
wherein the first drop-down connector member and the second drop-down connector member are configured to removably couple the second radiation attenuating material to the tray.

17. The radiation shield assembly of claim 16, wherein each of the first drop-down connector member and the second drop-down connector member comprise a hook configured to be secured to the second radiation attenuating material.

18. The radiation shield assembly of claim 16, further comprising a rod removably coupled to the first drop-down connector member and the second drop-down connector member, the rod being coupled to the second radiation attenuating material.

19. The radiation shield assembly of claim 16, wherein the first drop-down connector member comprises a first protrusion extending towards the second drop-down connector member, the second drop-down connector member comprises a second protrusion extending towards the first drop-down connector member, and the first protrusion and the second protrusion are configured to removably couple the second radiation attenuating material to the tray.

20. The radiation shield assembly of claim 1, further comprising an extension portion of the radiation attenuating material coupled to at least one of the first side, the second side, or the cranial side, the extension portion of the radiation attenuating material extending above the tray.
